(19) United States
(12) Patent Application Publication
(10) Pub. No.: US 2003/0064400 A1
(43) Pub. Date: Apr. 3, 2003

(54) Microfluidics system for single molecule DNA sequencing

(75) Inventor: John G.K. Williams, Lincoln, NE (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LI-COR, Inc., Lincoln, NE

(21) Appl. No.: 10/228,729

(22) Filed: Aug. 26, 2002

Related U.S. Application Data
(60) Provisional application No. 60/314,714, filed on Aug. 24, 2001.

(51) Int. Cl. C12Q 1/68; C12M 1/34

(52) U.S. Cl. 435/6; 435/287.2

(57) ABSTRACT

The present invention provides apparatus and methods for orientating a nucleic acid on a bead in a microchannel port. The microchannel system is designed to work with the transport and orientation characteristics of the bead to place the DNA into position for analysis. Preferably, orientation is achieved by a combination of flowcell architecture and energy fields.

### CROSS-REFERENCE TO RELATED APPLICATIONS

This application claims priority to U.S. Provisional Patent Application No. 60/314,714, filed Aug. 24, 2001, the contents of which are incorporated herein by reference in its entirety.

### BACKGROUND OF THE INVENTION

The primary sequences of nucleic acids are crucial for understanding the function and control of genes and for applying many of the basic techniques of molecular biology. The ability to do rapid and reliable DNA sequencing is therefore a very important technology. The DNA sequence is an important tool in genomic analysis as well as other applications, such as genetic identification, forensic analysis, genetic counseling, medical diagnostics, and the like. With respect to the area of medical diagnostic sequencing, disorders, susceptibilities to disorders, and prognoses of disease conditions, can be correlated with the presence of particular DNA sequences, or the degree of variation (or mutation) in DNA sequences, at one or more genetic loci. Examples of such phenomena include human leukocyte antigen (HLA) typing, cystic fibrosis, tumor progression and heterogeneity, p53 proto-oncogene mutations and ras proto-oncogene mutations (see, Gyllensten et al., PCR Methods and Applications, 1: 91-98 (1991); U.S. Pat. No. 5,578,443, issued to Santamaria et al.; and U.S. Pat. No. 5,776,677, issued to Tsui et al.).

Various approaches to DNA sequencing exist. The dideoxy chain termination method serves as the basis for all currently available automated DNA sequencing machines. (see, Sanger et al., Proc. Natl. Acad. Sci., 74: 5463-5467 (1977); Church et al., Science, 240: 185-188 (1988); and Hunkapiller et al., Science, 254: 59-67 (1991)). Other methods include the chemical degradation method, (see, Maxam et al., Proc. Natl. Acad. Sci., 74: 560-564 (1977), whole-genome approaches (see, Fleischmann et al., Science, 269, 496 (1995)), expressed sequence tag sequencing (see, Velculescu et al., Science, 270, (1995)), array methods based on sequencing by hybridization (see, Koster et al., Nature Biotechnology, 14, 1123 (1996)), and single molecule sequencing (SMS) (see, Jett et al., J. Biomol. Struct. Dyn. 7, 301 (1989) and Schecker et al., Proc. SPIE-Int. Soc. Opt. Eng. 2386, 4 (1995)).

U.S. Pat. No. 6,255,083, issued on Jul. 3, 2001 to Williams, and incorporated herein by reference, discloses a single molecule sequencing method on a solid support. The solid support is optionally housed in a flow chamber having an inlet and outlet to allow for renewal of reactants that flow past the immobilized polymerases. The flow chamber can be made of plastic or glass and should either be open or transparent in the plane viewed by the microscope or optical reader. Electro-osmotic flow requires a fixed charge on the solid support and a voltage gradient (current) passing between two electrodes placed at opposing ends of the solid support. The flow chamber can be divided into multiple channels for separate sequencing.

Other micro flow chambers exist. For example, Fu et al. Nat. Biotechnol. (1999) 17:1109 describe a microfabricated fluorescence-activated cell sorter with 3 μm×4 μm channels that utilizes electro-osmotic flow for sorting. In addition, U.S. Pat. No. 4,979,824, describes that single molecule detection can be achieved using flow cytometry wherein flowing samples are passed through a focused laser with a spatial filter used to define a small volume.

In addition, U.S. Pat. No. 4,793,705 describes a detection system for identifying individual molecules in a flow train of the particles in a flow cell. The patent further describes methods of arranging a plurality of lasers, filters and detectors for detecting different fluorescent nucleic acid base-specific labels.

Moreover, single molecule detection on solid supports is described in Ishikawa, et al. Jan. J Apple. Phys. 33:1571-1576. (1994). As described therein, single-molecule detection is accomplished by a laser-induced fluorescence technique with a position-sensitive photon-counting apparatus involving a photon-counting camera system attached to a fluorescence microscope. Laser-induced fluorescence detection of a single molecule in a capillary for detecting single molecules in a quartz capillary tube has been described. The selection of lasers is dependent on the label and the quality of light required.

Diode, helium neon, argon ion, argon-krypton mixed ion, and Nd:YAG lasers are useful in this invention (see, Lee et al. (1994) Anal. Chem., 66:4142-4149).

A need currently exists for a more effective and efficient flowcell for single molecule detection. These and further needs are provided by the present invention.

### BRIEF SUMMARY OF THE INVENTION

In certain aspects, the present invention provides apparatus, systems and methods for orientating a nucleic acid on a solid phase (e.g., a bead) in a microchannel port. The microchannel system is designed to work with the transport and orientation characteristics of the bead to place the DNA into position for analysis. Preferably, orientation is achieved by a combination of flowcell architecture and an energy field.

In one embodiment, the present invention provides a device for single molecule detection, comprising: a first substrate having a first channel disposed therein, the first channel having an inlet port and an outlet port; a second substrate having a second channel disposed therein, the second channel having an inlet port and an outlet port, wherein the first channel of the first substrate and the second channel of the second substrate intersect to form a fluidly connected presentation area; and an energy field applied across the first and second substrate to immobilize a solid phase (e.g., a bead) in the presentation area.

In another embodiment, the present invention provides an immobilized single molecule nucleic acid composition, comprising: a single nucleic acid immobilized on a solid phase, wherein the ratio of the immobilized single molecule nucleic acid to the solid phase is exactly 1.

In yet another embodiment, the present invention provides a device for single molecule detection, comprising: a substrate having a channel disposed therein, the channel having a port; and a single nucleic acid immobilized on a solid phase, wherein the single nucleic acid immobilized on a solid phase is trapped in the port.

In another embodiment, the present invention provides a method for separating a doubled end-labeled nucleic acid, the nucleic acid having a 3′ end adapter and a 5′ end adapter, the method comprising: ligating a first binding member to the 3′ end adapter to form a 3′ end label; ligating a second binding member to the 5′ end adapter to form a 5′ end label; providing a solid phase having a complementary binding member to the 3′ end label to form a first binding pair; and complexing a complementary binding member to the 5′ end label to form a second binding pair, thereby separating a doubled end-labeled nucleic acid.

These and other aspects and advantages of the present invention will become more apparent when read with the drawings and detailed description, which follows.

### BRIEF DESCRIPTION OF THE DRAWINGS

FIG. 1 illustrates a schematic of an embodiment of flowcell of the present invention.

FIG. 2 illustrates a schematic of an embodiment of a flowcell according to the present invention.

FIGS. 3A-F illustrate an immobilized single molecule nucleic acid composition, with a single-stranded DNA.

### DETAILED DESCRIPTION OF THE INVENTION

In one embodiment for single molecule DNA sequencing, the target nucleic acid molecule is placed in a flowcell where it can be analyzed. This is conveniently done by first attaching an individual DNA molecule to a bead. The bead is then placed in the flowcell by lodging at a constriction in the channel. There are many ways to form constrictions in a microchannel to capture a bead. Preferably, only 1 bead is trapped at any given position. Moreover, the bead should be trapped in such a way as to allow the captured nucleic acid molecule to be supplied with reagents for sequencing. A preferred configuration will maximize the number of sequencing channels (beads) in the optical field of view on the microchannel substrate. Described herein are systems, apparatus and methods for trapping individual beads that meet all of these requirements and additionally compatible with electrosorting methods described in U.S. application Ser. Nos. 09/876,374, and 09/876,375, both filed Jun. 6, 2001, the disclosures of which are hereby incorporated by reference in their entireties. The systems, apparatus and methods are useful for other approaches to single-molecule DNA sequencing where beads are employed for single-molecule placement in a microchannel.

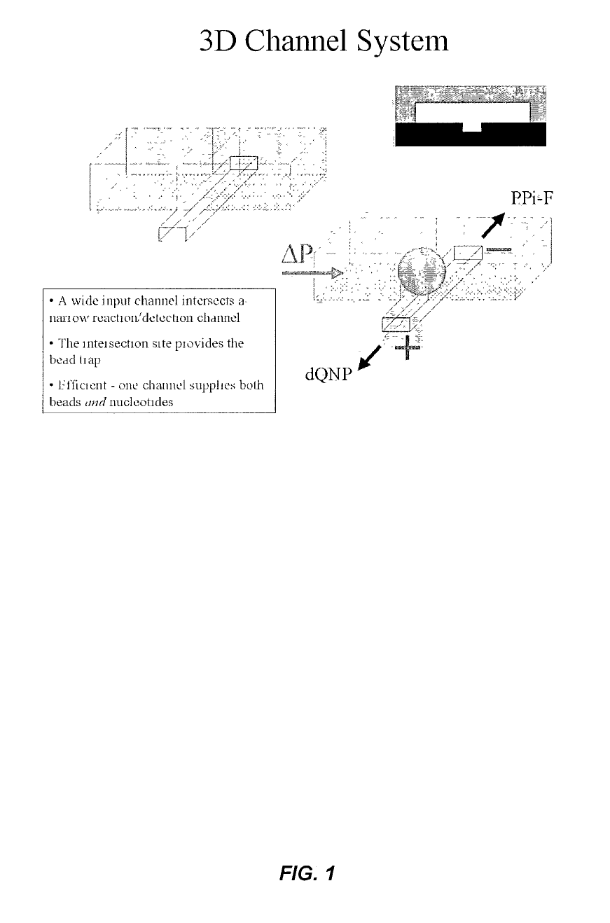

In one embodiment, a large microchannel is etched in a first substrate and a small microchannel is etched in a second substrate. The substrates can be made of fused silica, glass, or polymers such as polydimethyl siloxane (PDMS) or polymethyl methacrylate (PMMA). The two substrates are pressed together so that the two channels intersect at an orthogonal or oblique angle. In one preferred aspect, the center axes of the two channels are in different planes but they are fluidly connected at the intersection. The large channel is sufficiently large in cross section to permit passage of a micron-sized bead attached to a DNA molecule. The small channel is too small to permit passage of the bead. Preferably there is one DNA molecule attached to the bead for single-molecule analysis, but multiple molecules could be attached to enable simultaneous analysis of multiple DNA molecules for applications other than single-molecule DNA sequencing. An energy field (e.g., a pressure field or electric field) is applied across the two substrates so that the field lines pass from the large channel into the small channel and a bead driven by the field in the large channel is forced toward the small channel at the intersection. The bead is trapped at the intersection because it cannot fully enter the small channel. Preferrably, the bead does not completely block fluid flow through the intersection because the bead is round, the intersection cross-section is rectangular, and the large channel is slightly larger than the bead diameter to permit the bead to move through the large channel. The DNA on the trapped bead is also acted upon by the energy field so that it enters the second channel (providing that the second channel is sufficiently large to accept the DNA, at least 1.5 nanometer across, more preferably 0.1 micron, and most preferably at least 0.5-1.0 micron). The DNA is now positioned in the small channel, anchored to the bead trapped at the intersection (FIG. 1).

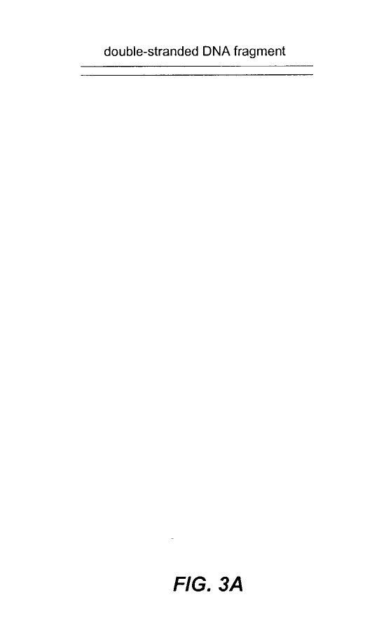

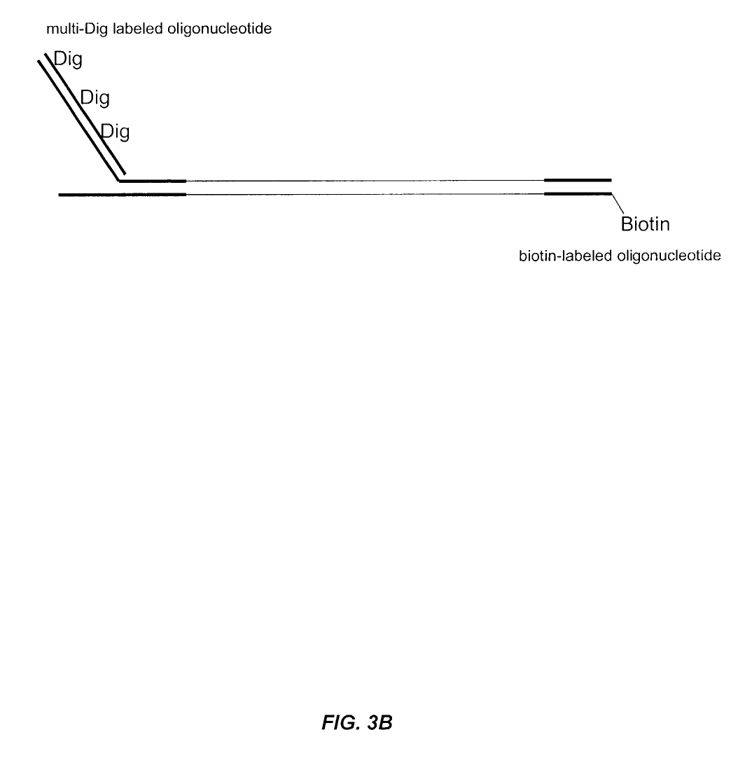

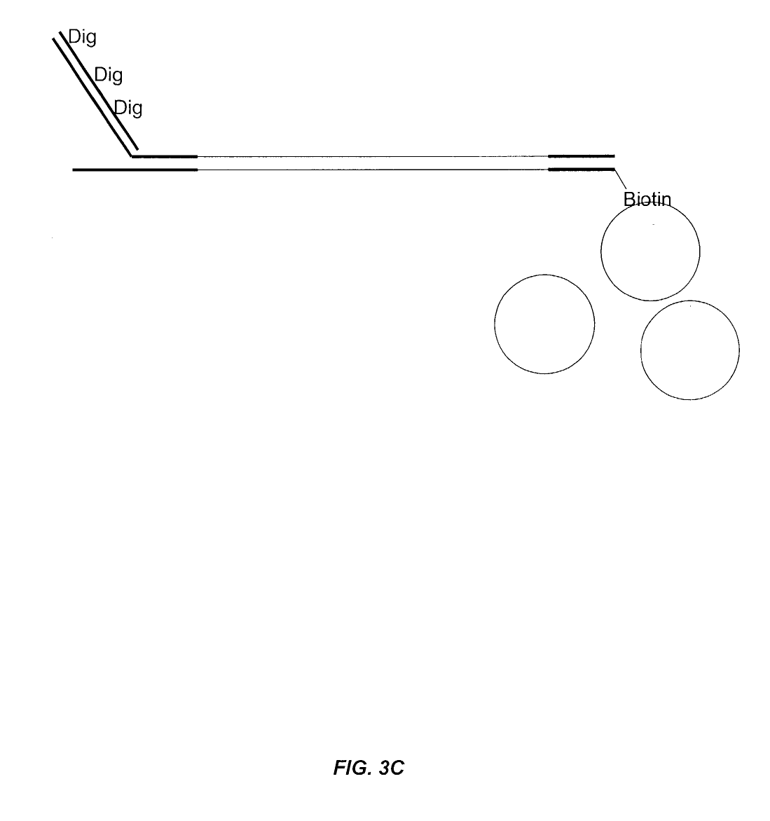

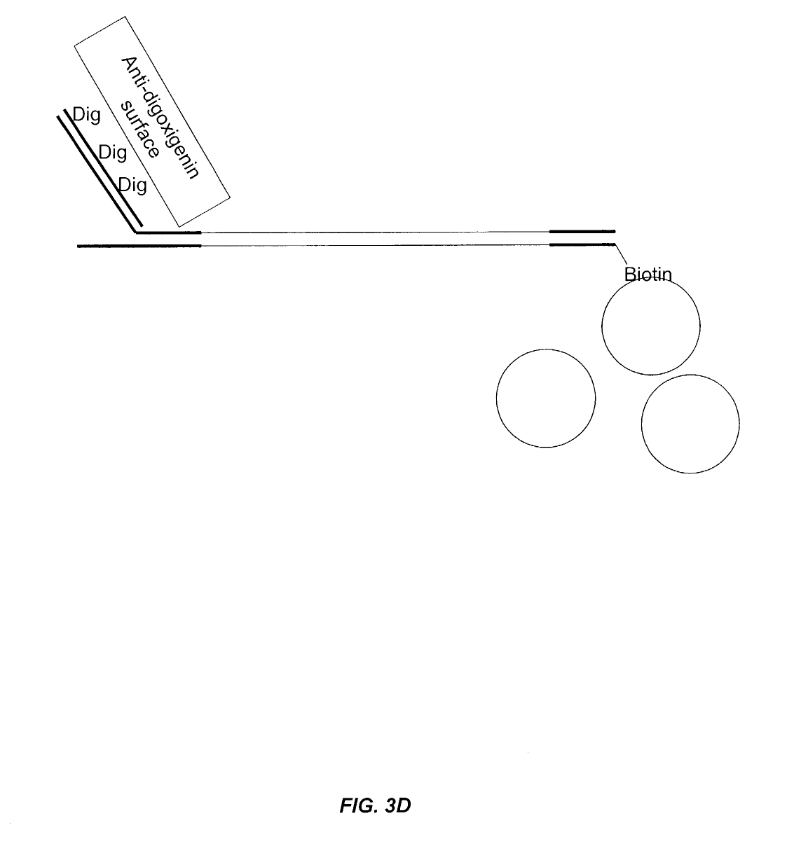

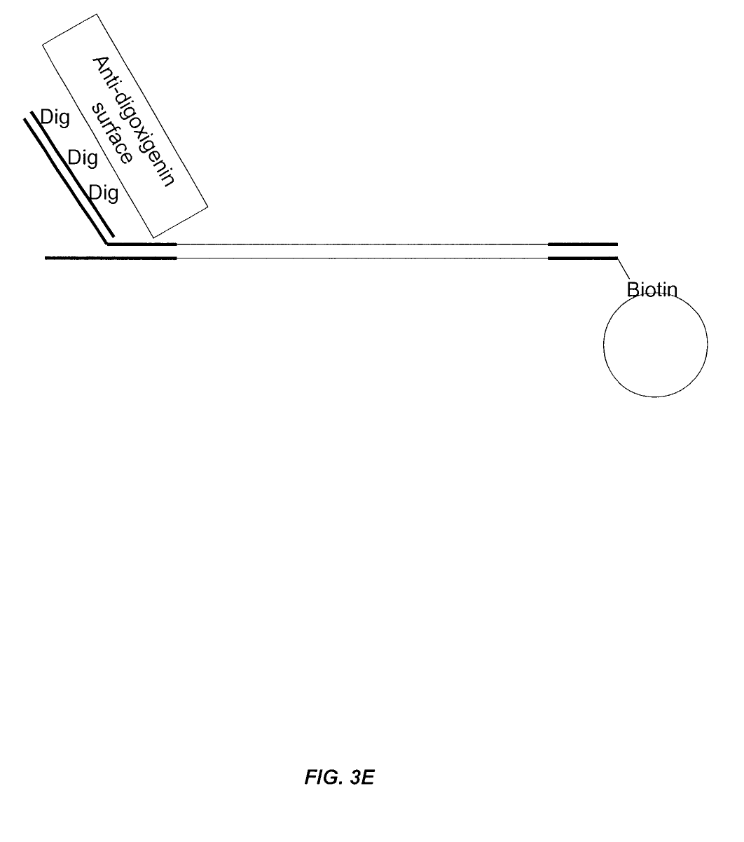

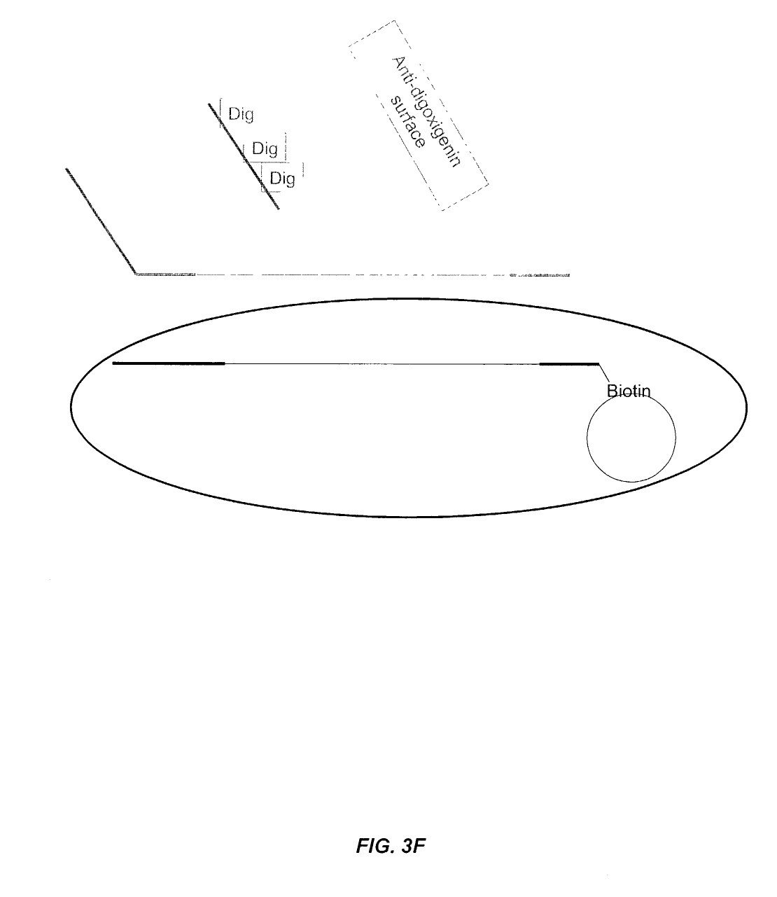

With reference to FIGS. 3A-F, in one embodiment, single DNA molecules are attached to a solid substrate. Double-stranded DNA fragments are prepared for attachment by shearing or by enzymatic cleavage from larger DNA molecules isolated from a source of interest (FIG. 3A). In the first process step, DNA fragments are end-labeled, by ligating (e.g., simultaneously) to two different oligonucleotide adapters, where the first adapter is labeled with a first ligand (e.g., biotin) and the second adapter is labeled with a second ligand (e.g., digoxigenin) (FIG. 3B). The adapters can be ligated by methods known in the art, for example, by first using t4 DNA polymerase to make the DNA fragment ends blunt and then using high concentrations of t4 DNA ligase to join the oligonucleotides to the ends of the DNA fragment.

If the two oligonucleotide ligands are biotin (B) and digoxigenin(D), respectively, for example, then ligation yields a mixture of DNA fragment types that differ in end-label composition (first end-second end): B-B, D-D, B-D, B-x, D-x, x-x , where x indicates an absence of label. The DNA fragments can then be purified (e.g., electrophoretically, chromatographically, filtration, and the like) to remove unligated oligonucleotide adapters. In the second process step, beads are attached to single DNA molecules (FIG. 3C). DNA fragment types BD, BB and Bx attach to streptavidin-coated magnetic beads under conditions of concentration and time where only some of the beads conjugate to a DNA fragment while most of the beads fail to conjugate. For example, at 1% coupling efficiency, 0.99% of the beads will have one DNA fragment while only 0.005% of beads will have more than one fragment (Poisson statistics). The coupling reaction is stopped by separating beads from unattached DNA fragments (e.g., using a magnet), allowing recovery of fragment types B-B, B-D and B-x on beads while discarding fragment types D-D, D-x and xx. The third process step purifies beads carrying single DNA molecules of type BD only; beads with other fragment types (B-B or B-x) are eliminated, as are beads without DNA. This is accomplished by binding beads to a surface coated with anti-digoxigenin antibodies (FIG. 3D). Unbound beads are washed away (FIG. 3E) and the bound beads are released by denaturing the DNA at high pH (FIG. 3F); the beads are released with their attached DNA fragments in single-stranded form, and the beads are separated from the other released molecules using a magnet. It will be appreciated by those skilled in the art that ligand-binder pairs other than biotin-streptavidin and digoxigenin-antidigoxigenin can be used in a similar manner. Binding can be enhanced by using multiple ligands on the DNA as shown in FIG. 3B for digoxigenin.

FIG. 3F shows that the oligonucleotide adapters remain attached to the isolated single DNA molecules. To sequence this DNA, an oligonucleotide primer is hybridized to the adapter sequence (e.g., to the bead-distal adapter in FIG. 3F if the strand is 3′-5′ from the free end of the DNA toward the bead-attached end of the DNA). The first few nucleotides sequenced from the 3′-end of the primer are determined by the sequence of the oligonucleotide adapter. The expected sequence can be used for quality control in the sequencing reaction; the first few nucleotides should be as expected if the sequencing system is performing properly. Moreover, if two different DNA samples are prepared on beads as described in the present invention, and if different oligonucleotide adapter sequences are used for the two samples, then the samples can be mixed together after the oligonucleotide adapters are ligated to the DNA. When the oligonucleotide adapters are sequenced during a sequencing run, the oligonucleotide sequences identify whether the current DNA molecule being sequenced originated from the first or from the second sample. This feature can be extended, allowing multiple DNA sample beads (a thousand or more) to be mixed together for analysis, where individual beads are sequenced one at a time and the sample identity of each is known by the sequence of their respective oligonucleotide adapters. The identifying oligonucleotide sequence can be the initial oligonucleotide (the one that hybridizes to the primer) or the final oligonucleotide (the one on the end opposite to where the primer hybridizes).

As used herein, the term “binding pair” refers to first and second molecules or ligands that specifically bind to each other (e.g., and binding member).

“Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

Exemplary binding pairs or ligands include any haptenic or antigenic compound in combination with a corresponding antibody or binding portion or fragment thereof (e.g., digoxigenin and anti-digoxigenin; fluorescein and anti-fluorescein; dinitrophenol and anti-dinitrophenol; bromodeoxyuridine and anti-bromodeoxyuridine; mouse immunoglobulin and goat anti-mouse immunoglobulin) and nonimmunological binding pairs (e.g., biotin-avidin, biotin-streptavidin, hormone (e.g., thyroxine and cortisol)hormone binding protein, receptor-receptor agonist or antagonist (e.g., acetylcholine receptor-acetylcholine or an analog thereof), IgG-protein A, lectin-carbohydrate, enzyme-enzyme cofactor, enzyme-enzyme-inhibitor, and complementary polynucleotide pairs capable of forming nucleic acid duplexes) and the like.

As such, in one embodiment, the present invention provides a method for separating a doubled end-labeled nucleic acid, the nucleic acid having a 3′ end adapter and a 5′ end adapter, the method comprising:

ligating to the nucleic acid a 3′ end adapter having a first binding member to form a 3′ end label;

ligating to the nucleic acid a 5′ end adapter having a second binding member to form a 5′ end label;

providing a solid phase having a complementary binding member to the 3′ end label to form a first binding pair; and

complexing a complementary binding member to the 5′ end label to form a second binding pair, thereby separating a doubled end-labeled nucleic acid.

Suitable solid phase materials include, but are not limited to, controlled pore glass, a glass plate or slide, polymers, polystyrene, acrylamide gel, activated dextran wells, agarose, polyacrylamide, polystyrene, polyacrylate, hydroxethylmethacrylate, polyamide, polyethylene, polyethyleneoxy, copolymers of the foregoing, non-porous surfaces, a raised region, a dimple, a pin, a trench, a rod, a bead, a pin, an inner or outer wall of a cylinder, a microfluidic channel and an addressable array.

### EXAMPLES

### Example 1

This example illustrates single-molecule placement and electrosort sequencing

In the electrosorting method of sequencing (described in U.S. application Ser. Nos. 09/876,374, and 09/876,375) an electric field is applied along the length of the small channel. The DNA is negatively charged and so it strains toward the positive electrode. NP probes and polymerase are supplied to the intersection from the large channel. They are both negatively-charged, so both reagents follow the electric field lines around the trapped bead and into the small channel going toward the positive electrode. The strained DNA is bathed in the solution of NP probes and polymerase. Upon each incorporation event, a labeled pyrophosphate moiety PPi-F is cleaved from the incorporated NP probe and its electric charge changes to net positive. This charge switch causes the PPi-F to reverse direction in the small channel and move towards the negative electrode. The cationic PPi-F moves against the flow of anionic NP probes and polymerases as it passes through the intersection and continues toward the negative electrode. Once through the intersection, the PPi-F is free from the labeled NP probes and it is detected with single-molecule sensitivity by fluorescence.

### Example 2

This example illustrates multiple sequencing channels.

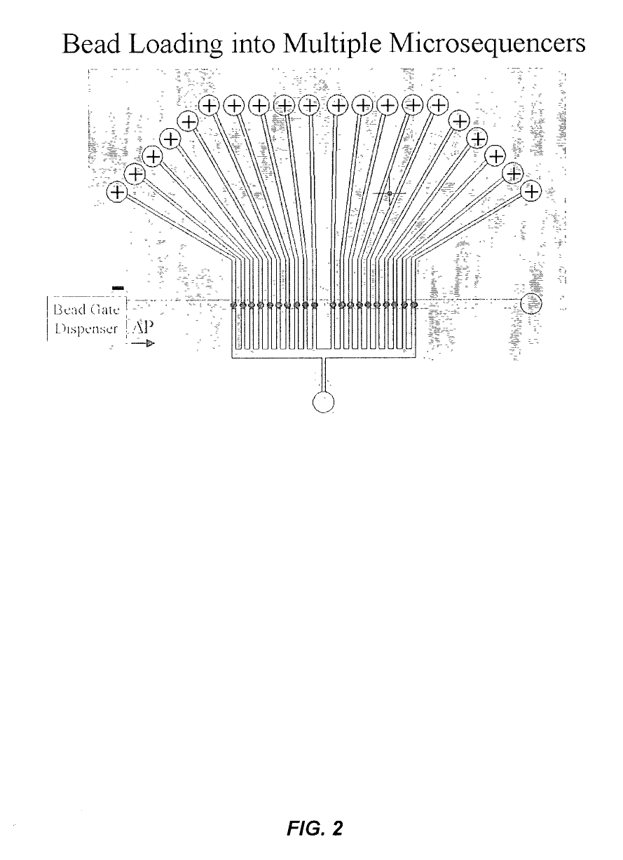

Multiple small channels can be arranged in parallel to so that many DNA molecules can be individually sequenced simultaneously. Preferably, beads are loaded one at a time by sequentially modulating the energy field in each small channel. The accuracy of the bead trapping can be monitored and controlled by observing each bead in real time (FIG. 2).

### Example 3

This example illustrates continuous processing using the apparatus and methods of the present invention.

When sequence analysis is complete, the energy fields can be reversed from the loading procedure and the trapped beads can be flushed out of the microchannel system through the large channel. Then fresh beads can be brought in and the process repeated continuously.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A device for single molecule detection, said device comprising:
a first substrate having a first channel disposed therein, said first channel having an inlet port and an outlet port;
a second substrate having a second channel disposed therein, said second channel having an inlet port and an outlet port, wherein said first channel of said first substrate and said second channel of said second substrate intersect to form a fluidly connected presentation area; and
an energy field applied across said first and second substrate to immobilize a solid phase in said presentation area.

2. The device for single molecule detection according to claim 1, wherein said solid phase has an immobilized single molecule nucleic acid on a bead and wherein the ratio of said immobilized single molecule nucleic acid to said solid phase bead is exactly 1.

3. The device for single molecule detection according to claim 1, wherein said first channel is wider than said second channel.

4. The device for single molecule detection according to claim 1, wherein said second channel is about 1.5 nm to about 1 μm wide.

5. The device for single molecule detection according to claim 1, wherein said energy field is selected from the group consisting of an electric, a magnetic hydrodynamic pressure field and combinations thereof.

6. The device for single molecule detection according to claim 1, wherein said second channel is an array of second channels.

7. An immobilized single molecule nucleic acid composition, said composition comprising:
a single nucleic acid immobilized on a solid phase, wherein the ratio of said immobilized single molecule nucleic acid to said solid phase is exactly 1.

8. The composition according to claim 7, wherein said immobilized single molecule nucleic acid occupies an area on said solid phase of greater than about 1 nm to about 1 μm.

9. The composition according to claim 8, wherein said immobilized single molecule nucleic acid occupies an area on said solid phase of greater than about 10 nm to about 100 nm.

10. The composition according to claim 7, wherein said solid phase is a member selected from the group consisting of controlled pore glass, polystyrene, a protein coated polystyrene bead, cellulose, nylon, acrylamide gel and activated dextran.

11. The composition according to claim 10, wherein said protein coated polystyrene bead is a member selected from the group consisting of an avidin coated polystyrene bead, a streptavidin coated polystyrene bead and mixtures thereof.

12. The composition according to claim wherein 11, said nucleic acid is a biotinylated DNA.

13. A substrate with a surface having a single nucleic acid with a known sequence bound to said surface at a density of 1 nucleic acid occupying a total area of greater than 100 μm² on said substrate with a ratio of substrate to nucleic acid of exactly 1.

14. A device for single molecule detection, said device comprising:
a substrate having a channel disposed therein, said channel having a port; and
a single nucleic acid immobilized on a solid phase, wherein said single nucleic acid immobilized on a solid phase is trapped in said port.

15. The device according to claim 14, wherein said port is smaller than the diameter of said bead trapped therein.

16. A method for separating a double end-labeled nucleic acid, said nucleic acid having a 3′ end adapter and a 5′ end adapter, said method comprising:
ligating to the nucleic acid a 3′ end adapter having a first binding member to form a 3′ end label;
ligating to the nucleic acid a 5′ end adapter having a second binding member to form a 5′ end label;
providing a solid phase having a complementary binding member to said 3′ end label to form a first binding pair; and
complexing a complementary binding member to said 5′ end label to form a second binding pair, thereby separating a double end-labeled nucleic acid.

17. The method of claim 16, wherein said complementary binding member to said 5′ end label is attached to a solid phase.

18. The method of claim 16, wherein said first binding member to said 3′ end adapter is selected from the group consisting of an antigenic compound, an antibody, a hormone, a receptor, an IgG-protein A, a carbohydrate, an enzyme, and polynucleotide.

19. The method of claim 16, wherein said first binding member to said 5′ end adapter is selected from the group consisting of an antigenic compound, an antibody, a hormone, a receptor, an IgG-protein A, a carbohydrate, an enzyme, and polynucleotide.

20. The method of claim 16, wherein solid phase is a member selected form the group consisting of controlled pore glass, a glass plate, a polymer, a raised region, a dimple, a pin, a trench, a rod, a bead, a pin, an inner or outer wall of a cylinder, a microfluidic channel and an addressable array.
